(19) United States
(12) Patent Application Publication
(10) Pub. No.: US 2026/0273310 A1
(43) Pub. Date: Sep. 17, 2026

(54) DEVICE AND METHOD FOR UPRIGHT ORBITAL RADIOTHERAPY

(71) Applicant: Cosylab d.d., Ljubljana (SI)

(72) Inventors: Matej Razpotnik, Novo mesto (SI); Zeljko Sljivic, Ljubljana (SI); Rok Stefanic, Ljubljana (SI); Andrej Vrecko, Radomlje (SI)

(21) Appl. No.: 19/474,566

(22) PCT Filed: Apr. 11, 2023
(86) PCT No.: PCT/EP2023/059464

§ 371 Date: Oct. 10, 2025

(51) Int. Cl. A61N 5/10

(52) U.S. Cl. CPC A61N 5/1081; A61N 5/1069; A61N 2005/1087; A61N 2005/1089; A61N 2005/109

(57) ABSTRACT

A device and method are provided for upright orbital radiotherapy. The device comprises a patient positioning system (PPS) for orienting a patient with respect to an irradiation device, the PPS arranged to be movable in at least one and up to two degrees of freedom (DOF), wherein at least one DOF is rotation around a vertical axis of the PPS. Further, an irradiation device is provided for emitting beams in a direction towards the PPS, the irradiation device being movable in at least 2 DOF and up to 5 DOF.

### CROSS REFERENCE TO RELATED APPLICATION

This present application is a § 371 national stage of International Application PCT/EP2023/059464, filed Apr. 11, 2023, which is hereby incorporated herein by reference in its entirety.

### FIELD

The present invention is in the field of medicine classified among devices for radiation therapy. The invention also belongs to the field of beam delivery systems. The invention relates to a device and a method for upright orbital radiotherapy.

### BACKGROUND

Radiation therapy is a type of cancer treatment that uses external beams of intense energy to kill cancer cells. Ionizing radiation damages the DNA of cancerous tissue affecting cellular growth and division leading to cellular death. To ensure as limited side effects as possible, shaped radiation beams are aimed from several angles of exposure to intersect at the tumour, providing a much larger absorbed dose there than in the surrounding healthy tissue. During the therapy, a margin of healthy tissue around the tumour is defined to allow for uncertainties in daily set-up and internal tumour motion caused by internal movement (for example, respiration and bladder filling) and movement of external skin marks relative to the tumour position. For radiation therapy mostly X-rays are used, however, protons or other types of irradiation sources are also common.

There are different types of external radio therapy (RT) systems available on the market, i.e., supine RT systems, upright RT systems and robotic arm RT systems and others.

The standard radiation therapy systems are based on complex supine configurations in which the radiation source rotates around the patient lying in the supine configuration targeting the prescribed patient position. Dominant devices are C-arm gantry-based systems like VersaHD from Electa and gantry-based system like Halcyon from Varian. These gantry-based systems are expensive to install and maintain. It is furthermore difficult to enable stable positioning (technical problem of gantry sag) and due to the rotatable X-ray source covering the whole 360-degree range a well-shielded room is needed, presenting additional cost for setting up a radiotherapy system which altogether results in high installation costs presenting a limiting factor for radiotherapy devices accessibility. Gantry-based systems also exhibit a limited field size, which is an additional constraint in the case of obese patients. U.S. Pat. No. 10,632,326B2 and patent application WO2022136839 disclose gantry-based systems that address the possibility of non-coplanar delivery and non-coplanar delivery together with the possible translation of irradiation source along the radius of the system, such as in patent EP2665519B1, making such solutions more technically demanding and by that even more expensive to install and maintain.

Robotic arm-based radiotherapy systems like CYBERKNIFE® from Accuray Incorporated as described in U.S. Pat. No. 8,655,429B2 use a radiation source mounted on the robotic arm enabling up to 6 degrees of freedom (DOF). Such systems enable the delivery of beams from multiple out-of-plane orientations (non-coplanar delivery) and are not limited to a single plane as in most gantry-based systems. As one of the primary goals in radiation therapy is delivering a highly conformal dose distribution to the target volume this also requires the delivery of non-coplanar beams which are provided by such robotic arm solutions. One of the drawbacks of existing robotic arm solutions is the weight and size constraints for the load exerted on the robotic arm, resulting in a small field size (from 5 to 120 mm at 80 cm source to axis distance) leading to limited use, and high system cost. In addition, a significant disadvantage of the CYBERKNIFE® system is that due to robot-arm implementation some components, such as the beam shaping device, have been simplified consequently sacrificing the performance of the irradiation source.

Furthermore, robotic arm solutions exhibit additional concerns related to safety increasing the QA requirements due to the large extent of motion freedom. The CYBERKNIFE® RT system includes a costly patient positioning system providing 5-DOF of motion capabilities or even a robotic patient positioning system exhibiting 6-DOF.

Upright radiotherapy treatment has been known for a long time; however, no successful commercial solutions have been available on the market until recently when Leo Cancer Care started marketing its upright-based system for radiotherapy and proton therapy. The solution is based on the upright positioning system described in U.S. Pat. No. 11,529,109B2 exhibiting movement in 6-DOF, thus enabling isocentric positioning of the patient's tumour relative to a fixed beam delivery system. This emerging technology enables more comfortable positioning for the patient and reduced motion in the pelvic, abdominal, thoracic, and head and neck regions. Although the entire RT solution from Leo Cancer Care together with a simplified shielding presents reduced expense and technical complexity as compared to supine gantry-based systems there is still a need for further reducing the cost and complexity of the whole RT system as well as the complexity of its installation and maintenance as the chair-based patient positioning system is either in a pit or needs a raised floor.

U.S. Pat. No. 11,529,109 discloses a patient positioning assembly for orienting a patient with respect to a radiation source. The patient positioning assembly is movable in a vertical direction, rotatable about a vertical axis, and tiltable. The patient support assembly is configurable between a first orientation, which sustains the patient in a seated position, and a second orientation, which sustains the patient in a generally standing position. The patient support assembly further comprises a shin rest adjustable in position by horizontal or substantially horizontal translation, and wherein, in the second orientation, the shin rest is positioned against shins of the patient. The patient support assembly may also comprise a seat member, and in the first orientation, the seat member is positioned against buttocks of the patient for sustaining the patient in the seated position. The seat member is adjustable in angular orientation such that, in the second orientation, the seat member is rotated downward and is positioned posteriorly of a thigh of the patient. The patient support assembly may additionally comprise arm rests and foot braces for further stabilization of the patient.

Based on the vertical patient positioning, Leo Cancer Care develops a real-time, image-guided photon radiotherapy treatment system known under the name Ruby. Photon radiation treatments are delivered in the upright orientation which comes with several benefits for treatment accuracy and improved patient experience. This solution utilizes patient rotation, a fixed beam, and a shield. A control system controls imaging, positioning and treatment delivery. However, the patient positioning system is demanding from the operational and technical point of view, wherein the construction of the patient positioning system due to movement in many DOF is challenging.

### SUMMARY

Alternative devices for upright radiation therapies address the shortcomings as well as complexity of the presently known solutions. The alternative devices reduce complexity of the upright RT system as well as enable an increased range of the relative positioning of the patient with respect to the irradiation beam source. Furthermore, the upright RT systems enable a range of non-coplanar delivery angles without sacrificing the performance of the irradiation source, and without the need for additional QA requirements due to safety risks.

The device for upright orbital radiation treatment according to one aspect comprises a patient positioning system and an irradiation device. The irradiation device is designed to move in at least 2 DOF (x, γ) to compensate for target (usually tumour) location and up to 5 DOF (x, y, z, α, β) to enable additional functionalities, such as adjustable source to target distance (STD) and non-coplanar beams. The patient positioning system is designed to move in at least one and up to 2 DOF, wherein the first, essential DOF is rotation around γ axis and the second, optional DOF is vertical translation (z). In one embodiment, the source of irradiation is movable in at least 3 (x, y, z) and up to 5 DOF (x, y, z, α, β), and the patient rotation system rotates around γ axis only.

The device according to the present disclosure, having the above-described construction, allows a novel technique of radiotherapy in which the target does not need to be located in the isocentre as in currently known methods. Instead, the irradiation device is designed to follow a circular trajectory of the target in a patient as a full circle or at least a part of the circular trajectory, resulting in the same relative motion between the source and the target as compared to the conventional iso-centric approach. The device may also adjust the source to target distance by movement in the x axis during irradiation. This can be achieved with any known treatment planning system (TPS) to optimize the irradiation plan with adjustable source to target distance resulting in additional possibilities, e.g., continuous treatment field size and dose rate adjustment. This new technique is applicable to any type of irradiation source, such as X-rays, gamma rays, electrons, protons, etc.

These and other objects, advantages, purposes and features of the present invention will become apparent upon review of the following specification in conjunction with the drawings.

### BRIEF DESCRIPTION OF THE DRAWINGS

Embodiments will be described in further detail based on exemplary embodiments and figures, which show:

FIG. 1 A side view of the device according to a possible embodiment in an initial position

FIG. 2 A side view of the device according to a possible embodiment, wherein tilt of the irradiation device is indicated with dashed lines

FIG. 3 A top view of the device according to a possible embodiment, wherein the irradiation device installed on the holder is moved to the right along y axis in order to enable irradiation of the target located in the left part of the patient's body

FIG. 4 A top view of the device according to a possible embodiment, wherein rotation of the PPS is shown and STD is constant.

FIG. 5 A top view of the device according to a possible embodiment, wherein rotation of the PPS is shown and STD is variable.

FIG. 6 Schematic representation of the null reference point, the setup reference point and the offset vector

### DETAILED DESCRIPTION

An exemplary embodiment is shown in FIG. 1, which shows:
the patient positioning system (PPS) 1 for orienting a patient P with respect to an irradiation device 2, wherein the PPS 1 can be of any type of upright positioning, namely seated and/or perched, and/or standing, wherein the PPS 1 is movable in one or two DOF, wherein essential DOF is rotation of the positioning system around its vertical axis 11, which is enabled with a suitable mechanism,
the irradiation device 2 comprising:
   a source of irradiation 21 arranged to emit beams 21a in the direction towards the PPS 1,
   a beam shaping device 22 arranged to suitably shape the beams 21a emitted by the source of irradiation 21,
      wherein the beam shaping device 22 and source of irradiation 21 are mounted on a holder 3, wherein the beam shaping device 22 is installed in a manner to cross the path of the irradiation beams 21a emitted by the source of irradiation 21, for example the beam shaping device 22 may be installed on the holder 3, on a separate holder or directly on the source of irradiation 21, wherein the holder 3 and/or the irradiation device 2 or its individual components 21, 22 are movable in at least two DOF:
   x axis, in which the irradiation device 2 moves forwards and backwards relative to the PPS 1,
   y axis, in which the irradiation device 2 moves left and right relative to the PPS 1, and optionally in any of the following:
   z axis, in which the irradiation device 2 moves upwards and downwards relative to the surface on which the device is placed,
   α axis, in which the beam shaping device 22 rotates around its horizontal axis (x) with or without irradiation source 21 using suitable rotational movement means, and
   β axis, which is essentially tilting of the irradiation device 2.

Each of the components of the irradiation device 2 may be movable separately from other components. For example, the beam shaping device 22 rotates around its horizontal axis (x) without the source of irradiation beams 21. Alternatively, the holder 3 may be designed to allow rotation in the α axis.

If the irradiation device 2 is movable only in x and y axis and not in z-axis, the PPS 1 is movable along two DOF, wherein:
one DOF is rotational movement around its vertical axis (γ), and
the second DOF is the vertical translation of PPS (z).

Several different combinations are possible, namely:
the irradiation device 2 is movable in x, y and z axis and the PPS 1 is movable in a rotational manner around its vertical axis (γ), or
the irradiation device 2 is movable in x and y axis and the PPS 1 is movable in a γ and z axis, or
the irradiation device 2 is movable in x, y, z and α axis and the PPS 1 is movable in γ axis, or
the irradiation device 2 is movable in x, y, z and β axis and the patient positioning system is movable in γ axis, or
the irradiation device 2 is movable in x, y, z, α and β axis and the PPS 1 is movable in γ axis, or
the irradiation device 2 is movable in x, y and α axis and the PPS 1 is movable in γ and z axis, or
the irradiation device 2 is movable in x, y and β axis and the PPS 1 is movable in γ and z axis, or
the irradiation device 2 is movable in x, y, α and β axis and the PPS 1 is movable in γ and z axis.

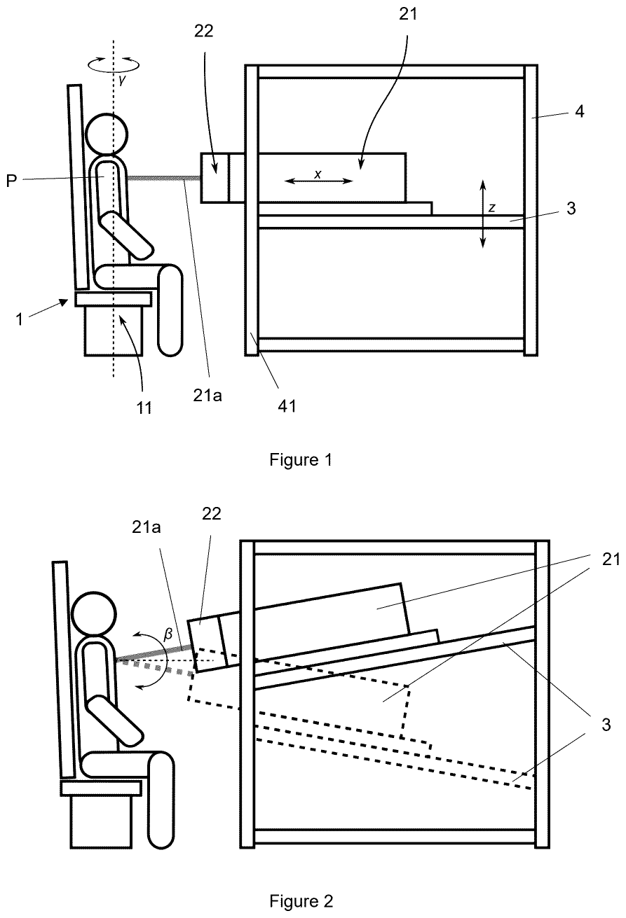

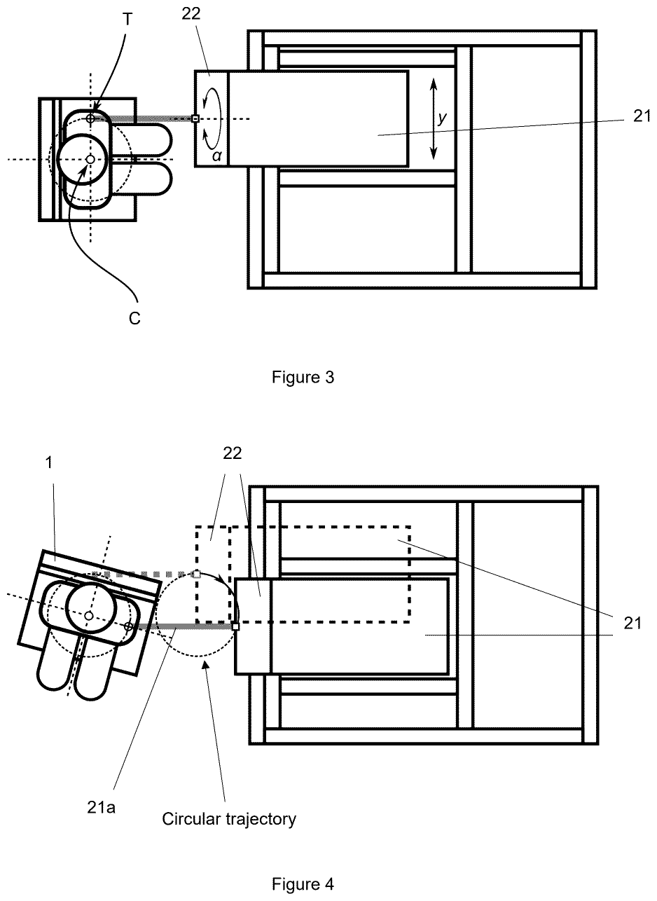

As one of the primary goals in radiation therapy is delivering a highly conformal dose distribution to the target volume this often requires the delivery of non-coplanar beams. Dashed lines in FIG. 2 indicate the tilt (β axis) of the irradiation device 2, while FIG. 3 shows movement of the irradiation device 2 in y axis to the right, so that the beam 21a targets the target T in the patient P positioned on the PPS 1. The centre C of rotation axis of the PPS 1 is also shown. Furthermore, FIG. 3 indicates rotation of the beam shaping means in α axis. In case tilting of the source of the irradiation (β axis) is enabled with the holder, a range of non-coplanar delivery angles is allowed. Furthermore, rotation (α) of the beam shaping device around its horizontal axis (x) additionally improves conformity of the dose distribution.

The movement in x, y and z axis, together with PPS's 1 rotation, enables motion of the irradiation device 2 relative to circular movement of the target T, while rotation around α axis allows higher modulation of the irradiation beams, i.e., different beam shapes, larger field size, etc. Movement in x axis allows a variable source to target distance (STD). Rotation around β axis enables non-coplanar irradiation, i.e., the tumour can be accessed from different angles and directions, thus the heathy tissues receive a smaller radiation dose, while most of the radiation is directed on the unhealthy tissue.

One embodiment of described device is arranged for translation of the source of irradiation in all three axes (x, y, z). Besides enabling a radiation beam to follow the trajectory of a target through translatory movement in an x-y plane such configuration exhibits a source of irradiation height (z) adjustment enabling treatment of different tumour locations without the need for increased upright positioning system complexity. The orbital movement of the tumour is exemplary shown in FIG. 4, wherein the irradiation device 2 emits a beam 21a directed to the patient P positioned in the PPS 1. The tumour is exemplary located in the left part of the patient P. The PPS 1 rotates and the tumour consequently describes at least a part, optionally full circular trajectory CT1. During this movement, the irradiation device 2 describes a circular trajectory CT2 during which a pre-planned (pre-defined) dose from the irradiation device is delivered in order to cause cellular damage in the tumour tissue.

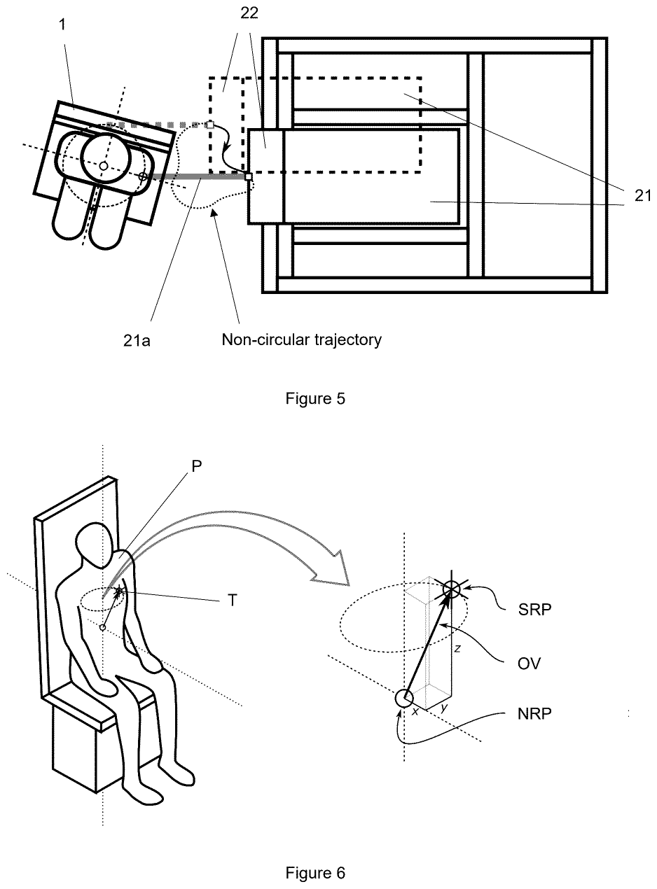

The device may also enable the variability of STD as mentioned above. By increasing the STD, the treatment field size becomes larger whereas decreasing the STD can be beneficial for target treatment where smaller field sizes can yield better results than larger fixed STD. Decreasing the STD furthermore increases the effective dose rates resulting in faster treatments. Furthermore, the varying STD during rotation of the PPS 1 supports arbitrary, not necessarily circular trajectories of the irradiation device, where trajectories can be either partial or full, as shown in FIG. 5.

The source of irradiation 21 and beam shaping device 22 may be mounted on the holder 3, which is used to allow movement of the irradiation device (FIGS. 1 to 3). In some embodiments, the holder 3 is a platform, a plate or a carrier grid installed on or in a supporting construction. Movement of the holder 3 in any of the defined DOF is achieved by any of the known manners. In general, any movement is enabled with at least one motor, which is controlled with a suitable controller that can be programmed to perform suitable movement of the driven axis with regards to the radiotherapy plan. The holder 3 may be supported by guides, grooves or any other guiding means along which the platform, plate or grid is movable on the supporting construction. Position sensors, shock absorbers and other regularly used elements are also provided.

In some examples, the holder designed as the carrier platform, the plate or the grid is adapted to tilt or pivot relative to a horizontal plane of the holder as shown in FIG. 2. The holder may be pivotally mounted to a translatable member such that the irradiation device may be tilted, or pivoted, about a point of the platform, plate or the grid. The point of tilting may be a centre point of the platform, plate or grid, or any other point located within an area defined by the platform, the plate or the grid. For example, the platform, the plate or the grid may be mounted onto a rounded member, which may be in the shape of a sphere, a hemisphere, a portion of a sphere, or other rounded surface, such that the platform, the plate or the grid is able to tilt and rotate relative to the rounded member. This freedom of movement may be used for tilting the irradiation device, for example, for allowing non-coplanar irradiation. Actuators may be provided for achieving the orientation and magnitude of tilt of the platform, the plate or the grid relative to the rounded member.

The beam shaping device 22 may rotate around its own axis (α) using the optional rotational movement means. The axis of the beam shaping device 22 is defined as being the same as the axis of the beam 21a that passes through it. Therefore, when the beam shaping device 22 is rotated, it will rotate around the axis of the beam 21a that it is modifying. This means that the direction and/or shape of the beam 21a can be altered as the beam shaping device 22 is rotated. Any possible beam shaping device 22 may be used in the device, wherein its selection depends on the source of irradiation. For example, for X-rays the choice for beam shaping device comprises primary collimator, jaws, multi-leaf collimator or any kind of beam collimation system.

The PPS 1 can be designed as a rotating platform comprising any suitable rotation mechanism. The platform is provided with an arrangement allowing sitting and/or perched and/or standing position. The PPS 1 for orienting the patient P with respect to the irradiation device 2 optionally includes:
a patient support assembly allowing above-mentioned positions of the patient, the assembly being attached to the base rotating around its vertical axis (γ axis), driven by any suitable mechanism providing rotational motion,
optionally a mechanism for vertical movement (z axis) of the PPS 1 allowing arbitrary vertical position in the defined range. This mechanism can be designed in any suitable known manner providing translatory motion.

The device for upright orbital radiotherapy is arranged to follow circular tumour movement and performs controlled movements (circular or non-circular) of the irradiation device 2 to deliver suitable doses to the target T. Device movement control is achieved with a suitable controller arranged to control the motor(s) that move the irradiation device 2, the beam shaping means 22 or the holder 3 on which the irradiation device 2 with the beam shaping means 22 is installed as well as the PPS 1.

The source of irradiation 21 may be any source suitable in radiation therapy. Some of the possible irradiation beam sources are X-rays, gamma rays, electrons, protons, neutrons, any type of radioactive isotopes, e.g., Cobalt60. Optionally, a medical linear accelerator (LINAC) is used to emit high energy X-rays or electrons. The device also allows use of LINACs with higher energies to ensure deeper penetration into the tissue. Even if the LINAC is larger and heavier, the holder is sufficiently sturdy to allow its installation. This is in contrast to the CYBERKNIFE@, in which the robotic arm does not allow installation of heavy and large accelerators.

In one embodiment, the irradiation device 2 is at least partly enclosed in a housing or an enclosure 4, which covers the irradiation device 2 and the holder 3 from all sides, wherein only the side 41 facing the PPS 1 has an opening through which the irradiation device 2 can protrude. This housing 4 may also serve as an additional shielding in some other irradiation source applications for instance Cobalt 60.

A source shield may be used with particular types of sources. For example, Cobalt source has a shield covering it in its entirety, while LINACs have simpler shielding and/or smaller shielding to prevent the leakage from the LINAC source once it is turned on to irradiate.

A primary barrier shielding (not shown) is provided behind the PPS 1 in the direction of the beam 21a. This aspect is well known and any known suitable primary barrier shielding solution may be used.

The disclosed device also facilitates a method for upright orbital radiotherapy using the device as described above, wherein the method includes at least the following:
the irradiation device 2 moves in at least 2 translatory DOF (x, y) and up to 5 DOF (x, y, z, α, β, γ).
the PPS 1 moves in at least one DOF (γ axis) and the second (optional) DOF (z axis).

In one embodiment of the method the patient positioning system is moving in one DOF, which is rotation around its vertical axis (γ), and the irradiation device is moving in three DOF (x, y, z).

Optionally, the irradiation device can move also in any of the two additional DOF (α, β) to provide extra functionalities. This optional method comprises:
moving the irradiation device along the x, y, z-axis using the holder provided with suitable guides and movement means;
rotating (α) the beam shaping means with or without the irradiation device and/or holder around the horizontal axis (x) using suitable movement means, and/or
tilting (β) the irradiation device using suitable tilting means attached or incorporated in the holder.

In a further optional embodiment of the method the irradiation device moves in two DOF (x and y) and optionally in α and β axes. In this case the PPS needs to move along two DOF, i.e., γ and z axis.

A setup of the device for upright orbital radiotherapy is needed before a patient undergoes treatment. The setup is patient- and case-specific and comprises two distinct points:
A null reference point NRP, which is a coincidence point between the vertical axis of the PPS 1 and the beam 21a axis. Since the beam axis or the PPS can be adjustable in z direction, trained personnel (usually radiation therapist) define the beam axis height which is typically set in the vicinity of the tumour location T. The null reference point is also marked on the patient with the help of lasers or any other visual indicator. This point is defined at the very beginning, before the CT simulation is performed and is utilised as the starting point for the patient positioning at all upcoming treatment sessions.
A setup reference point SRP is a point that defines a location of a target (usually tumour). It is defined by trained personnel (usually medical physicist or dosimetrist), who creates a treatment plan.

A vector connecting the null reference point and the setup reference point is called an offset vector OV. FIG. 6 depicts the null reference point, the setup reference point and the corresponding offset vector. It is important to note, that the offset vector can be calculated by any treatment planning system (TPS) available on the market.

The workflow of a treatment session, performed by the device according to the present disclosure, includes the following steps:
a) the patient is positioned and immobilized on the PPS,
b) the patient setup as described in the paragraph above is performed resulting in the device being positioned in the defined null reference point,
c) any suitable imaging is performed to obtain real-time data on the position and location of the tumour,
d) in case of a discrepancy, a correction vector is calculated and then added to the offset vector, wherein the correction vector is determined in any known manner known to the person skilled in the art,
e) a radiotherapy is performed as planned by the TPS, wherein;
   once the operator selected the beam, the irradiation device automatically moves for the offset vector possibly corrected with the correction vector,
   the PPS rotates and the irradiation device moves and delivers the beam to the target according to the selected beam in the treatment plan. As described above, the target T describes a circular movement, while the irradiation device 2 is arranged to follow various paths, either circular or non-circular.

The device allows variable STD and, as such, can perform additionally optimised treatment plans, which are even further tailored to the patient. This functionality can be achieved by a TPS that can handle variable STD.

The disclosed devices and methods have several advantages over prior art. Firstly, non-claustrophobic patient positioning compared to gantry-enclosed systems and easier accessibility for staff and patient. Additionally, the devices and methods allow variation of the distance between the irradiation source and the patient (source to target distance—STD), enabling treatment field size and dose rate adjustment. For a larger field size and thus larger area coverage, the STD is increased, whereas for a smaller field size, the STD is shorter, thus allowing treatment time reduction and reduced scattering. Finally, the principle of orbital tumour tracking allows higher accuracy of irradiation, as the device itself is more rigid and has much smaller sag compared to gantry systems. This system is also safer as there are fewer moving parts and collisions are not possible by design. Also, less shielding is needed. All these characteristics of the disclosed devices and methods lead to a more affordable device and thus improved access to radiation therapy treatments. Moreover, the devices and methods shorten the workflow as the radiation therapist does not have to perform positioning of the patient to move the setup reference point into the isocentre. This step is replaced by automatic and faster movement of the irradiation device.

Movement of the irradiation device is achieved in a substantially different manner compared to CYBERKNIFE® solution, since the present devices and methods do not have any compromises with regards to the irradiation source and the beam shaping means. As it is known, CYBERKNIFE® has weight and size constraints, resulting in a smaller field size and limited use. The present devices and methods allow various positions of the patient and the irradiation device, as well as dose modulation, shorter treatment times and increased usability in treating various tumours.

In comparison to solutions of Leo Cancer Care, the source of radiation beams is movable and the patient positioning system optionally moves in only one axis, which is rotation around its vertical axis. This configuration enables tumour positioning as well as tumour tracking without any other patient translation. Thus, this solution is simple and cost-effective, enables easy installation and maintenance and allows
non-coplanar beam delivery angles allowing intensity modulation and radiobiological optimization, thus simplifying the dose delivery and reducing the need for noncoplanar beam portals,
adjustable field size, as well as
increased range of patient positioning with respect to the source allowing to cover more tumour cases.

Changes and modifications in the specifically described embodiments can be carried out without departing from the principles of the present invention which is intended to be limited only by the scope of the appended claims, as interpreted according to the principles of patent law including the doctrine of equivalents.

1. A device for upright orbital radiotherapy, comprising:
an irradiation device;
a patient positioning system (PPS) for orienting a patient with respect to said irradiation device, said PPS arranged to be movable in at least one and up to two degrees of freedom (DOF), wherein at least one said DOF of said PPS is rotation around a vertical axis of the PPS; and
wherein said irradiation device is operable to emit irradiation beams in a direction towards the PPS, and said irradiation device is movable in at least two DOF and up to five DOF.

2. The device according to claim 1, wherein said PPS is arranged to allow an irradiated target to describe at least a part of a circular trajectory with respect to an axis of rotation of said PPS, and said irradiation device is arranged to describe at least a part of a circular or non-circular trajectory.

3. The device according to claim 1, wherein said irradiation device comprises:
an irradiation source arranged to emit irradiation beams in a direction towards said PPS; and
a beam shaping device installed in a manner to cross a path of the irradiation beams, wherein said beam shaping device is arranged to shape the irradiation beams emitted by said irradiation source;
wherein said beam shaping device and/or said irradiation source are mounted on a holder, wherein said holder and/or said irradiation source and/or said beam shaping device are movable in at least two DOF:
x axis, in which said irradiation device moves forwards and backwards relative to said PPS; and
y axis, in which said irradiation device moves left and right relative to said PPS.

4. The device according to claim 3, wherein said irradiation device is further movable in any of the following:
z axis, in which said irradiation device moves upwards and downwards relative to a surface on which said irradiation device is placed;
α axis, in which said beam shaping device is rotatably driven around its horizontal axis; and
β axis, which is essentially tilting of said irradiation device.

5. The device according to claim 3, wherein each of said irradiation source and said beam shaping device are movable independently of one another.

6. The device according to claim 3, wherein said beam shaping device is installed on said holder, on a separate holder or directly on said irradiation source.

7. The device according to claim 1, wherein
said irradiation device is movable in x, y and z axis and said PPS is movable in a rotational manner around its vertical axis (γ); or
said irradiation device is movable in x and y axis and said PPS is movable in γ and z axis; or
said irradiation device is movable in x, y, z and α axis and said PPS is movable in γ axis; or
said irradiation device is movable in x, y, z and β axis and said PPS is movable in γ axis; or
said irradiation device is movable in x, y, z, α and β axis and said PPS is movable in γ axis; or
said irradiation device is movable in x, y and α axis and said PPS is movable in γ and z axis; or
said irradiation device is movable in x, y and β axis and said PPS is movable in γ and z axis; or
said irradiation device is movable in x, y, α and β axis and said PPS is movable in γ and z axis.

8. The device according to claim 3, wherein said holder of said irradiation device is a platform, a plate or a carrier grid installed on or in a supporting construction.

9. The device according to claim 8, wherein movement of said holder is enabled with at least one motor controlled with a controller programmed to perform prescribed movement based on a radiotherapy plan set up by an operator of said device.

10. The device according to claim 8, wherein said holder is provided with:
position sensors;
shock absorbers; and
guides or grooves or guides along which said platform, plate or grid is movable in said supporting construction.

11. The device according to claim 8, wherein said holder is adapted to tilt or pivot relative to a horizontal plane of said holder.

12. The device according to claim 11, wherein said holder is mounted onto a rounded member, such that said platform, plate or grid is able to tilt and rotate relative to said rounded member, and wherein actuators are provided for controlling the orientation and magnitude of tilt of said holder relative to said rounded member in order to improve stability.

13. The device according to claim 1, wherein said PPS is arranged to allow a type of upright positioning of the patient selected from a group comprising seated, perched, and standing.

14. The device according to claim 1, wherein said PPS is designed as a rotating platform comprising a rotation mechanism.

15. The device according to claim 13, wherein said PPS comprises
a patient support assembly allowing the type of upright positioning of the patient, said patient support assembly being attached to a base rotating around its vertical axis.

16. The device according to claim 3, wherein said irradiation source is a therapeutic radiation source.

17. The device according to claim 3, wherein said irradiation source emits X-rays, gamma rays, electrons, protons, neutrons, or radioactive isotopes.

18. The device according to claim 3, wherein said irradiation source is a medical linear accelerator arranged to emit high energy X-rays or electrons.

19. The device according to claim 3, wherein said beam shaping device comprises primary collimator, jaws, multi-leaf collimator or any kind of beam collimation system.

20. The device according to claim 3, wherein said irradiation source is a linear accelerator shaped by a multi-leaf collimator.

21. The device according to claim 3, wherein
the irradiation device is at least partly enclosed in a housing or an enclosure (4), which covers said irradiation device and said holder from all sides, wherein only one side (41) facing said PPS has an opening through which said irradiation device can protrude; and
a primary barrier shielding is provided behind said PPS in the direction of the irradiation beams.

22. The device according to claim 3, wherein for setting up said device the following is determined:
a null reference point, which is a coincidence point between the vertical axis of said PPS and an axis of the irradiation beams; and
a setup reference point is a point that defines a location of a target;
wherein the null reference point and the setup reference point are connected with an offset vector.

23. A method of orbital treatment using the device according to claim 1, said method comprising:
a) positioning and immobilizing the patient on said PPS,
b) performing a patient setup by defining:
a null reference point which is a coincidence point between the vertical axis of said PPS and an axis of the irradiation beams; and
a setup reference point is a point that defines a location of a target;
wherein the null reference point and the setup reference point are connected with an offset vector;
c) positioning the device in the null reference point;
d) imaging to obtain real-time data on the position and location of a tumor;
e) in case of a discrepancy, calculating a correction vector and then adding the correction vector to the offset vector; and
f) performing a radiotherapy as planned by a treatment planning system, said performing the radiotherapy comprising:
selecting the irradiation beams;
after selecting the irradiation beams, automatically moving the irradiation device for the offset vector, wherein if there is a discrepancy the correction vector is first added to the offset vector; and
rotating the PPS and moving the irradiation device the irradiation beams to the target according to the selected irradiation beams.

24. The method according to claim 23, wherein an irradiation-source-to-target distance is adjustable by movement in an x axis during said performing the radiotherapy.

25. A method for positioning of the device for upright orbital radiotherapy according to claim 1, wherein said method comprises:
providing the irradiation device and the PPS;
moving the irradiation device in at least two translatory DOF (xy) and up to five DOF (x, y, z, α, β); and
moving the PPS in at least one DOF comprising rotation around the vertical axis of the PPS.

26. The method according to claim 25, wherein during said moving the PPS, said moving the irradiation device comprises moving the irradiation device in at least three DOF comprising:
x axis, in which the irradiation device moves forwards and backwards relative to the PPS;
y axis, in which the irradiation device moves left and right relative to the PPS; and
z axis, in which the irradiation device moves upwards and downwards relative to a surface on which the device is installed.

27. The method according to claim 23, wherein:
the irradiation device is moving along at least two DOF comprising:
x axis, in which the irradiation device moves forwards and backwards relative to the PPS; and
y axis, in which the irradiation device moves left and right relative to the PPS; and
PPS is moving along two DOF, wherein:
a first of the at least two DOF is rotational movement around a vertical rotational axis; and
a second of the at least two DOF is vertical translation of patient positioning system along z axis.
